(19) United States
(12) Patent Application Publication
(10) Pub. No.: US 2009/0169523 A1
(43) Pub. Date: Jul. 2, 2009

(54) HSC SELF-RENEWAL

(76) Inventors: Catherine Verfaillie, Leuven (BE); Helene Schoemans, Wezembeek Oppem (BE); Rikkert Snoeckx, Westerlo (BE)

Correspondence Address: SCHWEGMAN, LUNDBERG & WOESSNER, P.A., P.O. BOX 2938, MINNEAPOLIS, MN, 55402, US

(21) Appl. No.: 12/268,320

(22) Filed: Nov. 10, 2008

Related U.S. Application Data
(63) Continuation-in-part of application No. 11/452,081, filed on Jun. 13, 2006.
(60) Provisional application No. 60/690,089, filed on Jun. 13, 2005.

(51) Int. Cl. A61K 35/12; C12N 5/06; C12N 5/08; A61K 38/02

(52) U.S. Cl. 424/93.7; 435/377; 435/366; 514/12

(57) ABSTRACT

The invention is related to methods for culturing stem cells, more particularly hematopoietic stem cells (HSC). The invention relates to methods for HSC expansion and the use of RASSF8 to increase the retention and/or expansion of KLS cells in vitro. The invention is also directed to cells produced by the methods of the invention. The cells are useful, among other things, for treatment of disorders or diseases (e.g. leukemia). The invention also relates to the development of small molecules that may increase HSC self renewal in vitro and in vivo.

### RELATED APPLICATIONS/PATENTS

This application is a continuation-in-part of U.S. application Ser. No. 11/452,081 filed Jun. 13, 2006 and published as US/2007/0022482 on Jan. 25, 2007 which claims priority to U.S. Provisional Application Ser. No. 60/690,089, filed Jun. 13, 2005, which applications and publications are herein incorporated by reference.

### GOVERNMENT FUNDING

The invention described herein was developed with support from the National Institutes of Health grant number PO1-CA-65493-013. The U.S. Government has certain rights in the invention.

### FIELD OF THE INVENTION

The invention is related to methods for culturing somatic stem cells, more particularly hematopoietic stem cells (HSC). The invention relates to methods for HSC expansion and the use of RASSF8 (C12orf2) to increase the retention and/or expansion of HSC/KLS cells in vitro. The invention is also directed to cells produced by the methods of the invention. The cells are useful, among other things, for treatment of disorders or diseases (e.g. leukemia). The invention also relates to the development of small molecules that may increase HSC selfrenewal in vitro and in vivo.

### BACKGROUND OF THE INVENTION

Hematopoiesis is the process by which hematopoietic stem cells give rise to all hematopoietic lineages during the lifetime of an individual. To sustain life-long hematopoiesis, HSC must self-renew to maintain or expand the HSC pool, and they must differentiate to form committed hematopoietic progenitor cells (HPCs) that progressively lose self-renewal potential and become increasingly restricted in their lineage potential. A combination of extrinsic and intrinsic signals are thought to converge to regulate HSC differentiation versus self-renewal decisions, but the molecular mechanisms that regulate these processes are poorly understood.

A multitude of cytokines have been cloned that affect HSCs and HPCs; however, to date none of these, alone or in combination, can induce the symmetrical, self-renewing HSC division in vitro that is needed for HSC expansion. Recently, several novel regulators of HSC fate decisions have been identified. For instance, overexpression of HoxB4 results in expansion of murine and human HSCs with an increased competitive repopulation potential; novel extrinsic regulators implicated in self-renewal of HSCs include Notch, Wnt, and the morphogens, sonic hedgehog (Shh) and bone morphogenetic protein (BMP)-4. While the discovery of these novel regulators provides credence to the hypothesis that extrinsic and intrinsic signals can influence HSC fate, a more global gene and/or protein expression analysis of human HSC did provide additional preliminary insight into pathways that support HSC self-renewal (Eckfeldt et al., PloS, 2005, 3; 8:1449-1458). Since several diseases/disorders require a large amount of HSCs for transplantation and this HSC-availability is insufficient, there is an increased need for new methods to expand HSCs. Such methods would greatly facilitate the development of new therapies for hematopoietic disorders like leukemia for which currently no sufficient therapy exists. New methods for HSC-expansion would potentially overcome the problems with Graft Versus Host Disease (GVHD) complications correlated with the limited number of HSC currently available for transplantation, by making it possible to choose the best compatible donor, regardless of the amount of cells available (Schoemans H et al., Bone Marrow transplantation, 2006, 38(2): 83-93).

### SUMMARY OF THE INVENTION

One embodiment provides a method for maintaining/expanding stem cells in an undifferentiated state comprising culturing stem cells wherein RASSF8 is overexpressed or added as a protein or biologically active fragment or derivative thereof, or in the presence of a small molecule that activates the signal pathways activated by RASSF8. In one embodiment, RASSF8 is overexpressed or added for a period of about 1 day to about 14 days. For example, the period can be any amount of days including and between 1 and 14, such as 2, 3, 4, 5, 6, 7, 8, 9, 10, 11, 12, or 13 (or more).

In one embodiment, the stem cells are isolated from umbilical cord, bone marrow, placenta, muscle, endothelium, bone, central nervous system (CNS), liver, gastrointestinal track, lung, blood or skin. In another embodiment, the stem cells are HSCs. In another embodiment, the stem cells are mammalian, such as human.

Another embodiment provides the cells produced according to the methods described herein. One embodiment provides a pharmaceutical composition comprising the cells described herein.

One embodiment provides a method of treatment comprising administering a therapeutically effective amount of the cells described herein to a subject in need thereof. In one embodiment, the subject in need thereof is suffering from a non-malignant blood disorder, including, but not limited to, immunodeficiencies comprising SCID, fanconi's anemia, aplastic anemia, congenital hemoglobinopathy, or metabolic storage disease (Hurler's disease, Hunter's disease, or mannosidosis). In another embodiment, the subject is afflicted with cancer, including, but not limited to, hematological malignancies comprising acute leukemia, chronic leukemia (myeloid or lymphoid), lymphoma (Hodgkin's or non-Hodgkin's lymphoma), multiple myeloma, myelodysplastic syndrome, or non-hematological cancer (e.g., breast carcinoma, colon carcinoma, neuroblastoma, or renal cell carcinoma). In one embodiment, the subject is afflicted with a non-malignant blood disorder or cancer or has loss of somatic stem cells as a result of, for example, liver failure, emphysema, diabetes, burns or wounds.

In one embodiment, the subject has been treated with chemotherapy or radiation. In another embodiment, the subject has lost HSCs.

One embodiment provides a method of treatment comprising administering RASSF8 protein, a biologically active fragment or derivative thereof or a small molecule that activates the signal pathways activated by RASSF8 to a subject in need thereof so as to increase HSCs in the subject. In one embodiment, the subject is afflicted with a non-malignant blood disorder or cancer or has loss of somatic stem cells as a result of, for example, liver failure, emphysema, diabetes, burns or wounds.

Another embodiment provides the use of RASSF8 to maintain/expand stem cells in an undifferentiated state. Another embodiment provides the use/method of RASSF8 to increase differentiation via inhibition of RASSF8 expression and pathways.

### BRIEF DESCRIPTION OF THE FIGURES

FIG. 1: Expansion rates of untransduced, MIG transduced, and Rassf8-MIG transduced cells.

FIG. 2: Subpopulations of the cultured Lin− cells (8 days) based on flow cytometric analysis. After 8 days culturing in prestimulation medium, cells were stained with a cocktail of antibodies against the most important hematopoietic lineage markers. There is a significant increase in the total amount of KLS cells in the Rassf8-MIG transduced compared to MIG transduced and untransduced cells respectively (Rassf8-MIG vs. MIG: p=0.00282; Rassf8-MIG vs. untransduced: p=0.00293). Error bars indicate the standard deviation.

FIG. 3: Amount of CFC colonies relative to MIG-transduced cells (n=3). The amount of colonies between plated untransduced and MIG-transduced cells is not significantly different. The amount of colonies of Rassf8-MIG transduced cells however, is significantly lower compared to MIG-transduced cells in both amounts of plated cells (750: p=0.0081; 1500: p=0.011). Error bars indicate the standard deviation.

FIG. 4: Engraftment levels 4 weeks after transplantations. Every diamond represents engraftment data of 1 mouse. A significant difference in engraftment levels is seen after 4 weeks between Rassf8-MIG transduced cells when compared to engraftment levels from MIG transduced and untransduced cells (p<0.001).

FIG. 5: Engraftment levels 12 weeks after transplantation. Every dot represents engraftment data of 1 mouse. Also after 12 weeks, a significant difference in engraftment levels is seen between Rassf8-MIG transduced cells when compared to engraftment levels from MIG transduced and untransduced cells (p<0.001).

### DETAILED DESCRIPTION OF THE INVENTION

### Definitions

“A” or “an” means one or more than one.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning.

“Effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

The human sequence for RASSF8 cDNA and protein can be found at GenBank Accession number BC03002 and below:

| | | |
|---|---|---|
| 1 | agcggaggcg gcgcggggac gggcgctggg cggccgcgga gctccgggtg ccgccgcgtc | (SEQ ID NO:9) |
| 61 | cccagcgccc cggccggccc ctctgggcgg cctgcggggg cggcgcagtt gcgaaactga | |
| 121 | gtgcctgtgt agatcatcct agaatacctg tgtggtgctg tccttcctca agactacctc | |
| 181 | attctcactg gctcggagac tcctgcctgc tgagctgact acacagactt agtcttctcc | |
| 241 | actccgtgtt cctgcagcta gagacatgac ctaacaccct gatgaccact ctcagggacc | |
| 301 | ttgagtgact ggccggtgca ccatggaact taaagtatgg gtggatggag ttcagaggat | |
| 361 | tgtttgtgga gtcactgaag tcacaacttg ccaggaggtt gtcatagcct tagctcaagc | |
| 421 | aataggtcga actggaaggt acacccttat agagaaatgg agagatactg aaagacactt | |
| 481 | agcacctcat gaaaatccta tcatatcctt aaacaaatgg gggcagtatg ctagtgatgt | |
| 541 | gcagctcatt ctacgacgaa ctgggccgtc tctcagtgag cgacccactt cagacagtgt | |
| 601 | ggctcgaatt cctgaaagaa ctttatacag gcagagtctg ccccccttag ctaaactgag | |
| 661 | gcctcagatt gacaaatcaa tcaaaaggag ggaaccgaaa aggaaatcac tgacatttac | |
| 721 | aggaggtgcc aaaggattaa tggacatttt tggaaaaggt aaagaaactg agtttaagca | |
| 781 | aaaggtgctg aataactgca aaacaacagc agatgagttg aagaagctaa tccgtctgca | |
| 841 | gacagagaag cttcaatcca ttgagaaaca gctggaatct aatgaaatag aaataagatt | |
| 901 | ttgggagcaa aagtataatt ccaaccttga agaggaaatt gtccgtctag agcaaaagat | |
| 961 | caaaagaaac gatgtagaaa ttgaggagga agaattctgg gaaaatgaat tacagattga | |
| 1021 | acaggaaaat gaaaaacagc tgaaggatca acttcaagaa ataagacaga aaataacaga | |
| 1081 | atgtgaaaac aaattaaagg actatttggc acagatccgg actatggaaa gtggtcttga | |
| 1141 | agcagaaaaa ttgcaacggg aagttcaaga ggcacaggtc aatgaggaag aggttaaagg | |
| 1201 | aaagatcggt aaggtcaaag gggagattga cattcaaggc cagcagagtc tgaggttgga | |
| 1261 | aaatggcatc aaagctgtgg aaagatctct tggacaagcc accaaacgct tacaggacaa | |
| 1321 | agaacaggaa ctggagcagt tgactaagga gttgcggcaa gtcaatctcc agcagttcat | |
| 1381 | ccagcagaca gggacaaaag ttaccgtttt gccagcggag cccattgaaa tagaggcctc | |
| 1441 | acatgcagac attgaaaggg ggatcatcat tctttctgat aagcaggagt gtaaagatta | |
| 1501 | gatatcacac caaaagctca ggcaagaaaa gcaaaaatga acaagtggga ctacattaaa | |
| 1561 | cccaaaactc ctgcacaaca aaggaaacga tcaccaaaat gaaaaggtgg actgtagatt | |
| 1621 | gggagaaaat atttgcaaac cgttatctgg taggggttta atatccaaac tatataagga | |
| 1681 | actcacagaa cttaaagaaa acaaataatc caattaaaaa tggtcaaagg ggctgggcat | |
| 1741 | ggtggctcac gcctgtaatc tcagcacttt gggaggccga ggtgggagaa tcacaaggtc | |
| 1801 | aggagactga gaccatcctg gctaacacca tgatgggcta acacccatct ctactaaaaa | |
| 1861 | tacgaaacat tagccaggtg tggtggcacg cgcctgtaat cccagctact cgggaggctg | |
| 1921 | aggcagaagg attgcttgaa cccgggaggt ggaggttgca gtgagccgag atcgcattac | |
| 1981 | tgcactccag cctgggtaac agagtgagac tccatctcaa aaaaaaaaaa aaaatagata | |
| 2041 | tttttccaaa gaagacataa aaacagccaa caggtatatg aaaaagtgct caacatcact | |
| 2101 | gatcagcagg gaaatgcaaa tcaaagccac aatgagatac cacctcacac ctgttaggtt | |
| 2161 | acatctgcta ttgtcaaaaa gataagagat agcaagtgtc agcaagggtc tggagaaaag | |
| 2221 | ggaatttttg tatactgttg gtgggaatgt aaatcagtac ggctattgtg gaaaacagtg | |
| 2281 | tagaggtttc tcaaaaaatt aaaactatca tacaacccag caaaaaaaaa | |

| | |
|---|---|
| MELKVWVDGVQRIVCGVTEVTTCQEVVIALAQAIGRTGRYTLIEKWRDTERHLAPHENPI | (SEQ ID NO:10) |
| ISLNKWGQYASDVQLILRRTGPSLSERPTSDSVARIPERTLYRQSLPPLAKLRPQIDKSI | |
| KRREPKRKSLTFTGGAKGLMDIFGKGKETEFKQKVLNNCKTTADELKKLIRLQTEKLQSI | |
| EKQLESNEIEIRFWEQKYNSNLEEEIVRLEQKIKRNDVEIEEEEFWENELQIEQENEKQL | |
| KDQLQEIRQKITECENKLKDYLAQIRTMESGLEAEKLQREVQEAQVNEEEVKGKIGKVKG | |
| EIDIQGQQSLRLENGIKAVERSLGQATKRLQDKEQELEQLTKELRQVNLQQFIQQTGTKV | |
| TVLPAEPIEIEASHADIERGIIILSDKQECKD | |

The invention encompasses RASSF8 nucleic acid and/or protein and biologically active fragments and derivative thereof. For example, one embodiment includes RASSF8 nucleic acid and/or protein molecules that have about 50%, about 60%, about 70%, about 80%, about 85%, about 90%, about 92%, about 95%, about 98% or about 100% sequence identity to SEQ ID NO:9 or SEQ ID NO:10.

“Stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent.

A totipotent stem cell is a cell that can give rise to a new individual if provided with appropriate maternal support. After sperm fertilizes the egg, a zygote is formed which then has the potential to develop into a complete embryo. The fertilized egg and its immediate progeny during the blastula stage are totipotent stem cells, which means that each individual cell has the potential to create any type of cell necessary for embryonic development, included extraembryonic membranes and tissues required for mammalian development, the embryo itself, and all postembryonic tissues and organs. Approximately four days after fertilization, compaction and blastocyst formation occurs; within a blastocyst, the inner cell mass contains cells which are pluripotent as they can give rise to all cell types of the embryo proper, including somatic and germ cells. These cells are no longer totipotent as they cannot generate the trophectoderm. From this inner cell mass, embryonic stem (ES) cells can be generated. Pluripotency of ES cells is defined based on the following criteria: they generate embryoid bodies in vitro, can generate teratomas in vivo and when injected in the blastocyst, contribute to all somatic and germline cell types. These features are maintained by a set of transcription factors and genes that have recently been defined, including Oct3a/4, Nanog, Sox2, Essrb, Dppa4, Tcl1, and Tbx3. Embryonic development and the subsequent adult life are viewed as a continuum of decreasing potencies.

During gastrulation and subsequent developmental steps, pluripotent cells further specialize into multipotent stem cells, also termed adult stem cells. Adult stem cells are termed multipotent as they are committed to differentiate into the multiple cell types of a single tissue. For example, the hematopoietic stem cell (HSC)— the best studied multipotent stem cell —undergoes self-renewing cell divisions, if transplanted at the single cell level can give rise to all the lineages of the blood system and functionally repopulates the hematopoietic system of a myeloablated animal or human. More recently, tissue-specific stem cells have been isolated from many organs, including among others the central nervous system (CNS), epidermis, intestine, liver, lung, retina, and heart. Like HSCs, most of these organ-derived stem cells fulfill all the basic stem cell criteria: they are undifferentiated but tissue committed stem cells, they self-renew—albeit for many of these adult stem cells at a very slow rate, and generate within the tissue of origin differentiated progeny. As these adult stem cells have been more recently defined, less is known regarding their phenotype and function compared with HSCs.

Other cells termed unipotent stem cells also fulfill all criteria for stem cells except that they are able to contribute only to one mature cell type. In the category of unipotent, lineage-committed stem cells we can include, for example, corneal epithelial cells or endothelial progenitor cells muscle myosatellite cells and spermatogonial stem cells. Aside of unipotent spermatogonial stem cells, the isolation of new populations of adult spermatogonial stem cells from neonatal and adult testis have recently been reported. Such testis derived stem cells appear to be truly pluripotent, as they generate teratomas and contribute to somatic and germline lineages when injected in the blastocyst.

Somatic Stem Cells: Hematopoietic Stem Cells

Blood cell formation, also known as hematopoiesis, is a hierarchical process by which the hematopoietic stem cells (HSCs) give rise to committed hematopoietic progenitor cells (HPCs) that are capable of generating the entire repertoire of mature blood cells over the lifetime of an organism. HSCs, the pinnacle of the hematopoietic hierarchy, are functionally defined by their capacity for self-renewal, to maintain or expand the stem cell pool; multi-lineage differentiation, to generate and/or regenerate the mature lympho-hematopoietic system; and ultimately to home to the appropriate microenvironment in vivo where, through self-renewal and multi-lineage differentiation, they can restore normal hematopoiesis in a myeloablated host. As HSC differentiate they give rise to committed hematopoietic progenitor cells with limited self-renewal capacity and an increasingly restricted lineage potential. The earliest HSC cell-fate decision involves differentiation into either a common lymphoid or a common myeloid progenitor (CLP and CMP, respectively), establishing the major lymphoid and myeloid divisions of lympho-hematopoietic system. As the name implies, the CLP gives rise to the mature lymphoid B, T and NK cells; and the CMP gives rise to both megakaryocyte-erythrocyte progenitors (MEPs) and granulocyte-monocyte progenitors (GMPs) that further differentiate into the mature myeloid megakaryocytic, erythroid, granulocytic and monocytic lineages.

During human development hematopoiesis occurs in two distinct waves. The first wave, known as “primitive” hematopoiesis, arises in the extra-embryonic yolk sac beginning around 16 days of gestation and primarily gives rise to primitive nucleated red blood cells to facilitate oxygen delivery (RBCs). A subsequent “definitive” wave of hematopoiesis begins around 27 days of gestation with the appearance of long-term repopulating HSC capable of lympo-myeloid differentiation within the aorta/gonads/mesonephros (AGM) region of the embryo proper. These definitive HSCs migrate from the AGM and seed the fetal liver (FL), which is the major site of fetal hematopoiesis from six to 22 weeks. After 22 weeks, the FL HSC continue migrating, ultimately finding their way to the bone marrow microenvironment, the primary site of hematopoiesis into adulthood. While HSC from ontogenically distinct origins have unique functional attributes, they share the defining self-renewal and multi-lineage differentiation characteristics.

Throughout ontogeny, normal hematopoiesis requires a tightly regulated balance between HSC self-renewal and lineage differentiation to ensure the adequate production of mature blood cells while maintaining the HSC pool. The consequences of dysregulating this balance are exemplified in nature by aplastic anemia on one extreme, an exhaustion of the HSC pool resulting from lineage differentiation without adequate self-renewal, and on the other extreme by hematologic malignancies, clonal expansion of immature blood cells as a result of enhanced self-renewal of the stem cell itself, or from a more committed cell without terminal differentiation. The balance of HSC self-renewal and differentiation is influenced by the convergence of intrinsic cellular signals and extrinsic micro-environmental cues from the surrounding stem cell niche, but the specific signals that regulate HSC cell-fate decisions are only poorly understood.

The complexities of HSC development and the regulation of cell-fate decisions are of particular interest, as hematopoietic cell transplantation (HCT), also known as bone marrow transplantation (BMT), has been effectively used as a clinical therapy for almost four decades. HCT represents a mainstay of therapy for numerous hematologic and non-hematologic malignancies as well as non-malignant blood diseases, and HSCs also represent a vector for gene therapy applications. Standard HCT protocols involve ablation of a patient's failing or malignant hematopoietic system using radiation and/or chemotherapy followed by transplant of either bone marrow (BM) or granulocyte colony-stimulating factor (G-CSF) mobilized peripheral blood (MPB) from either the patient (autologous HCT) or a suitably histocompatibility antigen-matched (also know as HLA-matched) donor (allogeneic HCT) to restore normal hematopoiesis. The limited availability of suitably matched BM or MPB donors represents a major obstacle for HCT. As a result of this limitation, increasing attention has shifted toward developing alternative cell sources for HCT, and umbilical cord blood (UCB) has emerged as the primary candidate. UCB represents an ideal cell source for HCT for two main reasons. First, on a per cell basis HSCs from UCB have a greater in vitro proliferative potential and increased in vivo engraftment potential compared to HSCs from ontogenetically later sources (e.g. BM and MPB). Second, there is a lower than expected incidence of graft-versus-host disease compared to BM or MPB, a life-threatening complication following HCT, following UCB transplantation, likely due to the naïveté of the developing immune system. In spite of these advantages, the clinical utility of UCB as a graft is limited by the low and fixed number of HSCs present in available UCB units. Additionally, quiescent HSCs from UCB and other sources are refractory to gene delivery using standard oncoretroviral vectors that require cell division for efficient integration into the genomic DNA. Therefore, a major focus of experimental hematology is to develop conditions suitable for the ex vivo expansion of HSCs.

Phenotypic Characterization of Human HSCs

The functional analysis on specific hematopoietic cell populations has been facilitated by the development of both monoclonal antibodies that recognize cell-surface molecules expressed on distinct subsets of hematopoietic cells and high-speed fluorescence activated cell sorting (FACS) to prospectively isolate pure cell populations. Classically, selection for CD34⁺ cells has provided an effective means to enrich for in vivo repopulating human HSCs, but the recent identification of CD34⁻ in vivo repopulating human HSCs and evidence that CD34 expression is reversible on HSCs imply that this strategy for HSC enrichment isolates only a portion of human HSCs. Furthermore, fewer than 0.05% of CD34⁺ UCB cells are capable of long-term repopulation of the bone marrow of myeloablated xenogenic recipients, the gold standard assay for the enumeration of human HSCs. The purity of human HSCs can be further increased by isolating CD34⁺ cells that do not express CD38 and lineage-specific surface antigens, so called CD34⁺CD38⁻Lineage(Lin)⁻ cells, which comprise between 0.05-0.10% of UCB mononuclear cells. To date, the CD34⁺CD38⁻Lin⁻ fraction of UCB represents one of the purest populations of long-term in vivo repopulating HSCs, but it remains considerably heterogeneous as it contains fewer than 0.2% repopulating HSCs. Therefore, even highly-purified, phenotypically-identical subsets of human UCB cells contain considerable functional heterogeneity. In lieu of the ability to prospectively identify and isolate pure populations of human HSCs and HPCs, numerous in vitro and in vivo functional assays have been developed to measure the quantity and quality of human HSCs and HPCs contained within heterogeneous cell populations in a retrospective fashion.

In Vitro Analysis of Human HSCs and HPCs

While in vivo assays provide the most physiologically relevant context for the functional characterization of human hematopoietic cells, the ability to analyze the functional attributes of limiting numbers of cells, or even single cells, under defined conditions makes in vitro assays an invaluable tool. The developmental continuum of hematopoiesis begins with HSCs, cells with tremendous self-renewal and multi-lineage differentiation potential, that give rise to committed HPCs, endowed with an increasingly limited capacity for self-renewal and restricted lineage differentiation potential, that ultimately give rise to terminally differentiated blood cells, incapable of either self-renewal or further differentiation. Therefore, hematopoietic cells can be characterized and classified in vitro based on their capacity for self-renewal and the lineage diversity of their progeny. Short-term in vitro assays, as their name implies, are designed to enumerate lineage-restricted HPCs that have a very limited self-renewal potential and are capable of differentiation into mature hematopoietic cells within two to three weeks in response to appropriate cytokine stimulation after minimal HPC division. Short-term in vitro assays specifically detect lineage-restricted myeloid HPCs, but not HSCs or primitive HPC subsets that are incapable of generating colonies in these protracted two to three week cultures. The standard assay for the enumeration of lineage-restricted HPCs is the colony-forming cell (CFC) assay in which cells are suspended in a semi-solid methylcellulose matrix culture system containing granulocyte colony stimulating factor (G-CSF), granulocyte-monocyte colony stimulating factor (GM-CSF), stem cell factor (SCF), interleukin 3 (IL3), and erythropoietin (Epo) to promote myelo-erythroid differentiation. Under these conditions, lineage-restricted HPCs give rise to distinct colonies, so called colony-forming units in culture (CFU-C), that can be enumerated to determine CFU-C frequency. The resulting colonies can be categorized based on colony size, morphology, and cell composition into colony-forming unit granulocyte-monocyte (CFU-GM), colony-forming unit erythroid (CFU-E), burst-forming unit erythroid (BFU-E), and multi-lineage colonies represented by colony-forming unit mix (CFU-mix), comprised of both granulocyte-monocyte and erythroid progeny, or colony-forming unit granulocyte-erythrocyte-monocyte-megakaryocyte (CFU-GEMM) comprised granulocyte, monocyte, erythroid and megakaryocyte progeny. Additionally, the relative maturity of CFU-Cs can be assessed using the CFC assay. Unlike more mature CFU-Cs, more primitive CFU-Cs are larger, in most cases containing multiple differentiated cells types, and can generate secondary colonies when re-plated in fresh CFC assays (secondary CFU-Cs), a reflection of their increased proliferative potential. While a majority of short-term hematopoietic progenitor assays exclusively detect the presence of myeloid progenitors, clonogenic in vitro assays to detect the presence of a short-term myeloid/lymphoid progenitors capable of generating B cells, T cells, and myeloid cells from murine fetal liver and the presence of murine pre-B cell precursors have also been described.

In vitro assays have also been developed to evaluate the presence of more primitive subsets of hematopoietic progenitor cells that likely represent an intermediate level of maturity between long-term HSC and short-term CFU-Cs, so called blast colony-forming cells (blast-CFCs) and high proliferative potential-colony-forming cells (HPP-CFCs). Blast-CFCs represent a rare population of cells that are capable of forming colonies of 40-1,000 small refractile cells that lack signs of terminal differentiation in clonogenic assays and are capable of giving rise to multi-lineage CFU-GEMM colonies in secondary CFC assays with a near 100% re-plating efficiency. HPP-CFC represent another class of primitive hematopoietic progenitors that are capable of generating large (>0.5 mm) monocyte/macrophage colonies after 14 days in semi-solid medium. Characteristic of primitive hematopoietic cell populations, both blast-CFCs and HPP-CFC share a relative resistance to 5-FU toxicity, a reflection of their quiescent nature relative to more mature CFU-Cs.

Primitive HPCs and presumed HSCs were originally detected in vitro in heterogeneous long-term bone marrow culture (LTBMC) capable of sustained production of hematopoietic progenitor cells in culture. Subsequently, conditions were defined that enabled the enumeration of primitive cells that are incapable of giving rise to colonies in short-term CFC, blast-CFC, or HPP-CFC assays, but generate clonogenic progenitor cells after extended culture periods (>5 weeks) that support the maturation and subsequent readout. One such long-term in vitro assay is the cobblestone-forming cell (CAFC) assay in which cells are maintained in liquid culture in contact with a monolayer of irradiated, or otherwise mitotically inactivated, stromal cells that partially recapitulate the complex in vivo bone marrow microenvironment to support HSC maturation. Long-term CAFCs are cells capable of generating a cobblestone-like colony of flattened cells in close association with the supporting stromal layer after five or more weeks in culture, signifying the long-term survival and proliferative potential of these primitive cells, defining characteristics of primitive hematopoietic cells. Another long-term in vitro assay is the long-term culture initiating cell (LTC-IC) assay that also assesses the long-term survival and proliferative potential of primitive hematopoietic cells after 35-60 days (standard LTC-IC) or 60-100 days (extended (e)LTC-IC) in liquid co-cultures containing supportive stromal cells. In contrast to the CAFC assay, the LTC-IC assay read-out is the ability of cells maintained for >5 weeks in liquid culture to generate one or more secondary CFU-Cs when placed in CFC conditions. LTC-IC assays are commonly performed using bulk cell populations to provide a crude measure of the total number of CFU-C generated per input cell. However, LTC-IC assays performed in this way cannot enumerate input LTC-IC frequencies, as a single LTC-IC can give rise to multiple CFU-C. Although it has been postulated that LTC-IC generate on average 4 CFU-Cs, differing culture conditions may affect the number of secondary CFU-Cs per input LTC-IC. Hence to enumerate the exact input number of LTC-ICs, standard limiting dilution experiments and Poisson statistics are used. Combining bulk and limiting dilution experiments, the number of CFU-C per input LTC-IC can be determined, providing both LTC-IC frequency and a measure of the generative potential of LTC-IC. While it has been speculated that LTC-IC represent a more primitive subset of CAFC that retain clonogenic potential after long-term culture, there is currently little data to support this hypothesis. The validity of the use of in vitro assays to enumerate cells with primitive hematopoietic function is supported by the strong correlation between the frequency of murine LTC-IC and CAFC with in vivo repopulating cells and data demonstrating a moderate to high correlation of human LTC-IC frequencies and in vivo repopulation potential for freshly isolated hematopoietic cells. However, human cells capable of in vivo repopulation in xenogenic transplant models have a much lower frequency than in vitro LTC-IC, and they are not maintained to the same degree as LTC-IC during ex vivo expansion cultures. These differences likely result from differences in the efficiency of detecting primitive cells using in vitro versus in vivo assays as well as differences in the functional requirements for in vitro defined HSC versus in vivo defined HSC.

HSCs are endowed with the capacity to generate not only myeloid progeny, but also lymphoid progeny. Thus, a major shortcoming of long-term in vitro HSC assays, such as the LTC-IC assay, is that they exclusively assess the generation of myeloid progeny, and thus may overestimate true HSC frequencies. The first in vitro system developed to evaluate the long-term development of lymphoid progeny was the Whitlock-Witte culture system, in which LTBMC conditions were optimized to support the development heterogeneous populations of pre-B, immature B, and mature B lymphocytes. To more closely recapitulate the in vivo differentiation programs of HSCs in vitro, assays that assess both long-term myeloid and lymphoid potential of human hematopoietic cells were subsequently developed. In the lymphoid-myeloid “switch culture” single cells are seeded on S17 stromal cells that primarily support the generation of CD19⁺ B-cell progenitors, and following proliferation during a primary expansion culture, the progeny of the input cell are evaluated for myeloid potential, determined by expression of CD33 and/or CFC formation, in secondary switch cultures. The myeloid-lymphoid initiating cell (ML-IC) assay also enumerates single cells that are capable of giving rise to at least one long-term myeloid progenitor and one long-term lymphoid progenitor in vitro. Neither the switch culture or ML-IC assay is suitable for evaluating the T lymphoid potential of input progenitor cells, as in vitro T cell development requires complex interactions with thymic stromal cells in three dimensional fetal thymic organ cultures (FTOC) or co-culture with specialized transgenic stromal cells capable of supporting T lymphogenesis. Despite this caveat, the multi-potent progenitor detected in the switch culture and ML-IC assays likely represents a more primitive progenitor than LTC-IC, and more closely resembles the potential of true HSCs. Furthermore, input of single cells in the ML-IC assay enables the determination of both frequency and generative potential of input ML-IC in a single assay. Therefore, the switch culture and ML-IC assays represent the most robust in vitro human HSC assays.

In Vivo Analysis of Human HSCs and HPCs

In vitro assays, that evaluate the self-renewal and multi-lineage differentiation potential of input cells, have been an invaluable tool for the qualitative and quantitative analysis of human hematopoietic cells; however, true HSC and HPC must not only self-renew and differentiate in vitro, but must also be able to home to the appropriate microenvironment in vivo to restore normal hematopoiesis in a myeloablated recipient. While there are advantages to the defined conditions that are attainable in vitro, it is not possible to accurately recapitulate the complex three dimensional cell-cell and cell-matrix interactions of the bone marrow microenvironment in a two dimensional culture system. Therefore, in vivo assays provide the most rigorous systems for the evaluation of HSC and HPC function. While in vivo transplant of human HSCs into human recipients would provide the most physiologically relevant assay of HSC function, such experiments are not tenable on ethical grounds. Therefore, in vivo assays for human HSCs rely on xenogenic recipients to evaluate their in vivo potential. However, xenogeneic transplants are complicated by immune-mediated rejection of the transplanted human cells by recipient animals. As a consequence, in vivo assays have been developed using various immunocompromised animal models.

One of the most well established xenogeneic transplant recipients for human HSC is the nonobese diabetic-severe combined immunodeficiency (NOD-SCID) mouse, that has defects in innate immunity and harbors a mutation in the Prkdc gene, rendering it incapable of generating mature B and T cells and thus unable to effectively reject transplanted human cells. Long-term engraftment of human hematopoietic cells in the bone marrow of NOD-SCID mice, so called SCID-repopulating cells (SRC), requires the transplantation of hundreds to thousands of human cells for successful engraftment, and thus is not a clonal assay. As such, the presence of long-term repopulating HSCs with self-renewal and multi-lineage differentiation potential is inferred from the ability of engrafted cells to generate both myeloid and lymphoid lineages in primary transplant recipients for greater than six to eight weeks, the presumed lifespan of primitive HPCs. The clonal contributions of multi-lineage, long-term SRC in engrafted recipients can be established by retrovirally marking SRCs prior to transplant. In this way, integration of pro-viral DNA into the SRC genome serves as a molecular signature to identify the clonally related progeny of single SRCs, and thereby directly establishes the long-term, multi-lineage differentiation potential of single cells. Long-term self-renewal and multi-lineage differentiation potential of SRCs can be more rigorously evaluated by assessing engraftment of SRCs after extended periods of time (i.e. 12-14 weeks), or more robustly by performing serial transplants of SRCs into successive recipients. While retroviral marking of SRCs provides some information regarding the numbers of SRCs in a given cell population, the frequency of SRCs is more commonly determined by transplanting cells in limiting dilutions.

While the classic NOD-SCID xenotransplant model provides a robust assay for multi-lineage human HSC engraftment, its utility to investigate transplant immunology has been limited by the failure of human lymphoid progenitors to efficiently migrate to the murine thymic microenvironment to generate mature T cells. Therefore, alternative models have been exploited for studying the in vivo development of adaptive immunity. The SCID-hu thymus mouse contains fragments of human thymic tissue that are transplanted under the kidney capsule of SCID mice to provide a xenograft microenvironment for the development of human thymopoiesis. The Rag2^{-/-}γ_{c}^{-/-} mouse has a deletion of both the Rag2 DNA recombinase activity, precluding the development of mature B and T cells, and lacks the IL2 receptor 7 common chain, required for IL2, IL4, IL7, IL9 and IL15 signaling, resulting in profound adaptive and innate immunodeficiency. This double knock-out model facilitates the development of a functional adaptive immune system from human cells transplanted into the fetal liver. The NOD-SCID-γ_{c}^{-/-} mouse, harboring a γ common chain deletion on the NOD-SCID background has also proven an effective model to study human immunity. Therefore, selecting a suitable model for xenotransplant of human stem and progenitor cells is dictated by the particular progenitors and progeny that are of interest.

Pluripotent Stem Cells

“EC cells” were discovered from analysis of a type of cancer called a teratocarcinoma. In 1964, researchers noted that a single cell in teratocarcinomas could be isolated and remain undifferentiated in culture. This type of stem cell became known as an embryonic carcinoma cell (EC cell).

“Embryonic Stem Cells (ESC)” are well known in the art and have been prepared from many different mammalian species for many years. Embryonic stem cells are stem cells derived from the inner cell mass of an early stage embryo known as a blastocyst. They are able to differentiate into all derivatives of the three primary germ layers: ectoderm, endoderm, and mesoderm. These include each of the more than 220 cell types in the adult body. The ES cells can become any tissue in the body, excluding a placenta. Only the morula's cells are totipotent, able to become all tissues and a placenta.

“Induced pluripotent stem cells (IPSC)” or “IPS cells” are somatic cells that have been reprogrammed by introducing exogenous genes that confer on the somatic cell a less differentiated phenotype. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them.

“MAPC” is an acronym for “multipotent adult progenitor cell.” It refers to a non-embryonic stem cell that can give rise to cell lineages of all three germ layers (i.e., endoderm, mesoderm and ectoderm) upon differentiation. Like embryonic stem cells, human MAPCs express telomerase, Oct 3/4 (i.e., Oct 3A), rex-1, rox-1 and sox-2, and may express SSEA-4. The term “adult” in MAPC is non-restrictive. It refers to a non-embryonic somatic cell.

MAPCs constitutively express Oct 3/4 (i.e., Oct 3A) and high levels of telomerase (Jiang, Y. et al., Nature, 418:41 (2002); Exp Hematol, 30:896, 2002). MAPCs derived from human, mouse, rat or other mammals appear to be the only normal, non-malignant, somatic cell (i.e., non-germ cell) known to date to express very high levels of telomerase even in late passage cells. The telomeres are extended in MAPCs and they are karyotypically normal. Because MAPCs injected into a mammal can migrate to and assimilate within multiple organs, MAPCs are self-renewing stem cells.

“Multipotent,” with respect to MAPC, refers to the ability to give rise to cell lineages of more than one primitive germ layer (i.e., endoderm, mesoderm and ectoderm) upon differentiation, such as all three.

“Primordial embryonic germ cells” (PG or EG cells) can be cultured and stimulated to produce many different cell types.

“Self-renewal” refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has dedifferentiated, for example, by nuclear transfer, by fusions with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

“Subject” means a vertebrate, such as a mammal, such as a human. Mammals include, but are not limited to, humans, dogs, cats, horses, cows and pigs.

The term “therapeutically effective amount” refers to the amount determined to produce any therapeutic response in a mammal. For example, effective amounts of the therapeutic cells or cell-associated agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are ascertained by one of ordinary skill in the art through routine application to subject populations such as in clinical and pre-clinical trials. Thus, to “treat” means to deliver such an amount.

“Treat,” “treating” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

Methods and Compositions of the Invention

The methods of the invention preserves stem cells, more particularly HSCs, in culture preventing the differentiation process. Such culture methods can be used to expand/propagate these stem cells keeping them in an undifferentiated state.

Culture methods of the invention comprise an overexpression of RASSF8 or a part (e.g., a biologically active fragment) or derivative thereof, or a small molecule that activates the signal pathways activated by RASSF8

Stem Cells

The present invention can be practiced, preferably, using stem cells of vertebrate species, such as humans, non-human primates, domestic animals, livestock, and other non-human mammals.

In an embodiment of the invention, supplements to keep stem cells, more particularly HSCs, in an undifferentiated state are cellular factors like RASSF8 or components thereof that allow said stem cells to retain the ability to differentiate upon removal or inhibiting said supplements. This may be indicated by the expression of specific markers (known in the art) of the undifferentiated state. Cells with a cKit+lin−Sca1+ (KLS) phenotype for instance are enriched in undifferentiated pluripotent HSCs. MAPCs, for example, constitutively express Oct 3/4 (Oct 3A) and maintain high levels of telomerase. Assays for monitoring gene expression are well known in the art (e.g., RT-PCR) and can be conducted using standard methodology.

The present invention provides methods wherein constitutive overexpression of RASSF8 increases the retention/expansion of KLS cells in vitro, blocks differentiation of progenitors giving rise to CFC and inhibits engraftment of HSC. The present invention therefore provides new methods of HSC expansion comprising short term overexpression of RASSF8.

Cell Culture

In general, cells useful for the invention can be maintained and expanded in culture medium that is available to and well-known in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of hematopoietic cells SFEM®. Many media are also available as low-glucose formulations, with or without sodium pyruvate.

Also contemplated in the present invention is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements and bovine embryonic fluid. It is understood that sera can be heat-inactivated at 55-65° C. if deemed necessary to inactivate components of the complement cascade.

Additional supplements also can be used advantageously to supply the cells with the necessary trace elements for optimal growth and expansion. Such supplements include insulin, transferrin, sodium selenium and combinations thereof. These components can be included in a salt solution such as, but not limited to, Hanks' Balanced Salt Solution® (HBSS), Earle's Salt Solution®, antioxidant supplements, MCDB-201® supplements, phosphate buffered saline (PBS), ascorbic acid and ascorbic acid-2-phosphate, as well as additional amino acids. Many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Hormones also can be advantageously used in the cell cultures of the present invention and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine and L-thyronine.

Lipids and lipid carriers also can be used to supplement cell culture media, depending on the type of cell and the fate of the differentiated cell. Such lipids and carriers can include, but are not limited to, cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin and oleic acid unconjugated and conjugated to albumin, among others.

Also contemplated in the present invention is the use of feeder cell layers. Feeder cells are used to support the growth of fastidious cultured cells, such as ES cells. Feeder cells are normal cells that have been inactivated by γ-irradiation. In culture, the feeder layer serves as a basal layer for other cells and supplies cellular factors without further growth or division of their own (Lim, J. W. and Bodnar, A., 2002). Examples of feeder layer cells are typically human diploid lung cells, mouse embryonic fibroblasts and Swiss mouse embryonic fibroblasts, but can be any post-mitotic cell that is capable of supplying cellular components and factors that are advantageous in allowing optimal growth, viability and expansion of stem cells. In many cases, feeder cell layers are not necessary to keep ES cells in an undifferentiated, proliferative state, as leukemia inhibitory factor (LIF) has anti-differentiation properties. Therefore, supplementation with LIF can be used to maintain cells in an undifferentiated state.

Cells may be cultured in low-serum or serum-free culture medium. Serum-free medium used to culture cells is described in, for example, U.S. Pat. No. 7,015,037. Many cells have been grown in serum-free or low-serum medium. For example, the medium can be supplemented with one or more growth factors. Commonly used growth factors include, but are not limited to, bone morphogenic protein, basis fibroblast growth factor, platelet-derived growth factor and epidermal growth factor, Stem cell factor, thrombopoietin, Flt3Ligand and 1′-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for teaching growing cells in serum-free medium.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. Hematopoietic stem cells can also be cultured in low attachment flasks such as but not limited to Corning Low attachment plates.

Once established in culture, cells can be used fresh or frozen and stored as frozen stocks, using, for example, DMEM with 40% FCS and 10% DMSO. Other methods for preparing frozen stocks for cultured cells also are available to those skilled in the art.

Methods of identifying and subsequently separating differentiated cells from their undifferentiated counterparts can be carried out by methods well known in the art. Cells that have been induced to inhibit differentiation using methods of the present invention can be identified by selectively culturing cells under conditions whereby undifferentiated cells have a specific phenotype identifiable by FACS. Similarly, differentiated cells can be identified by morphological changes and characteristics that are not present on their undifferentiated counterparts, such as cell size and the complexity of intracellular organelle distribution. Also contemplated are methods of identifying differentiated cells by their expression of specific cell-surface markers such as cellular receptors and transmembrane proteins. Monoclonal antibodies against these cell-surface markers can be used to identify differentiated cells. Detection of these cells can be achieved through fluorescence activated cell sorting (FACS) and enzyme-linked immunosorbent assay (ELISA). From the standpoint of transcriptional upregulation of specific genes, differentiated cells often display levels of gene expression that are different from undifferentiated cells. Reverse-transcription polymerase chain reaction, or RT-PCR, also can be used to monitor changes in gene expression in response to differentiation. Whole genome analysis using microarray technology also can be used to identify differentiated cells.

Accordingly, once differentiated cells are identified, they can be separated from their undifferentiated counterparts, if necessary. The methods of identification detailed above also provide methods of separation, such as FACS, preferential cell culture methods, ELISA, magnetic beads and combinations thereof. One embodiment of the present invention contemplates the use of FACS to identify and separate cells based on cell-surface antigen expression.

Pharmaceutical Formulations

In certain embodiments, the purified cell populations, RASSF8 protein, biologically active fragments or derivatives thereof or small molecules are present within a composition adapted for and suitable for delivery, i.e., physiologically compatible. Accordingly, compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

In other embodiments, the purified cell populations are present within a composition adapted for or suitable for freezing or storage.

In many embodiments the purity of the cells for administration to a subject is about 100%. In other embodiments it is about 95% to about 100%. In some embodiments it is about 85% to about 95%. Particularly in the case of admixtures with other cells, the percentage can be about 10%-15%, about 15%-20%, about 20%-25%, about 25%-30%, about 30%-35%, about 35%-40%, about 40%-45%, about 45%-50%, about 60%-70%, about 70%-80%, about 80%-90%, or about 90%-95%. Or isolation/purity can be expressed in terms of cell doublings where the cells have undergone, for example, about 10-20, about 20-30, about 30-40, about 40-50 or more cell doublings.

The numbers of cells in a given volume can be determined by well known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The choice of formulation for administering the compositions of the invention for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Various embodiments of the invention comprise measures to increase cell survival and/or to overcome problems posed by barriers to seeding and/or growth.

Final formulations of the aqueous suspension of cells/medium, protein or small molecules will typically involve adjusting the ionic strength of the suspension to isotonicity (i.e., about 0.1 to 0.2) and to physiological pH (i.e., about pH 6.8 to 7.5. The final formulation will also typically contain a fluid lubricant, such as maltose, which must be tolerated by the body. Exemplary lubricant components include glycerol, glycogen, maltose and the like. Organic polymer base materials, such as polyethylene glycol and hyaluronic acid as well as non-fibrillar collagen, preferably succinylated collagen, can also act as lubricants. Such lubricants are generally used to improve the injectability, intrudability and dispersion of the injected material at the site of injection and to decrease the amount of spiking by modifying the viscosity of the compositions. This final formulation is by definition the cells, RASSF8 protein, biologically active fragments or derivatives thereof or small molecules in a pharmaceutically acceptable carrier.

The compositions are subsequently placed in a syringe or other injection apparatus for precise placement at the preselected site. The term “injectable” means the formulation can be dispensed from syringes having a gauge as low as 25 under normal conditions under normal pressure without substantial spiking. Spiking can cause the composition to ooze from the syringe rather than be injected into the tissue. For this precise placement, needles as fine as 27 gauge (200μ I.D.) or even 30 gauge (150μ I.D.) are desirable. The maximum particle size that can be extruded through such needles will be a complex function of at least the following: particle maximum dimension, particle aspect ratio (length:width), particle rigidity, surface roughness of particles and related factors affecting particle:particle adhesion, the viscoelastic properties of the suspending fluid, and the rate of flow through the needle. Rigid spherical beads suspended in a Newtonian fluid represent the simplest case, while fibrous or branched particles in a viscoelastic fluid are likely to be more complex.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or stabilizer can be employed to increase the life of cell/medium compositions. If such preservatives are included, it is well within the purview of the skilled artisan to select compositions that will not affect the viability or efficacy of the cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells/medium, protein or small molecules utilized in practicing the present invention in the required amount of the appropriate solvent with various amounts of the other ingredients, as desired.

In some embodiments, cells/medium, protein or small molecules are formulated in a unit dosage injectable form, such as a solution, suspension, or emulsion. Pharmaceutical formulations suitable for injection of cells/medium, protein or small molecules typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells, protein or small molecules and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments stem cells, protein or small molecules are encapsulated for administration, particularly where encapsulation enhances the effectiveness of the therapy, or provides advantages in handling and/or shelf life. Encapsulation in some embodiments where it increases the efficacy of cell mediated immunosuppression may, as a result, also reduce the need for immunosuppressive drug therapy.

Also, encapsulation in some embodiments provides a barrier to a subject's immune system that may further reduce a subject's immune response to the cells (which generally are not immunogenic or are only weakly immunogenic in allogeneic transplants), thereby reducing any graft rejection or inflammation that might occur upon administration of the cells.

Cells, protein or small molecules may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation that may be used for administration are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules). Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference.

Certain embodiments incorporate cells, protein or small molecules into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the invention, cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

Dosing

Compositions (e.g., compositions containing cells, small molecules, RASSF8 protein or fragments or derivatives thereof) can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of cells/medium, protein or small molecules appropriate to be used in accordance with various embodiments of the invention will depend on numerous factors. It may vary considerably for different circumstances. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration. Finally, the determination of optimal dosing necessarily will provide an effective dose that is neither below the threshold of maximal beneficial effect nor above the threshold where the deleterious effects associated with the dose outweighs the advantages of the increased dose.

The optimal dose of cells for some embodiments will be in the range of doses used for autologous, mononuclear bone marrow transplantation. For fairly pure preparations of cells, optimal doses in various embodiments will range from about 10⁴ to about 10⁸ cells/kg of recipient mass per administration. In some embodiments the optimal dose per administration will be between about 10⁵ to about 10⁷ cells/kg. In many embodiments the optimal dose per administration will be about 5×10⁵ to about 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, cells/medium, protein or small molecules may be administered in an initial dose, and thereafter maintained by further administration. Cells/medium, protein or small molecules may be administered by one method initially, and thereafter administered by the same method or one or more different methods. The levels can be maintained by the ongoing administration of the cells/medium, protein or small molecules. Various embodiments administer the cells/medium, protein or small molecules either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regimens can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer the cells/medium, protein or small molecules

In some embodiments cells/medium, protein or small molecules are administered to a subject in one dose. In others cells/medium, protein or small molecules are administered to a subject in a series of two or more doses in succession. In some other embodiments wherein cells/medium, protein or small molecules are administered in a single dose, in two doses, and/or more than two doses, the doses may be the same or different, and they are administered with equal or with unequal intervals between them.

Cells/medium, protein or small molecules may be administered in many frequencies over a wide range of times. In some embodiments, they are administered over a period of less than one day. In other embodiment they are administered over two, three, four, five, or six days. In some embodiments they are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

Additionally, RASSF8 protein, a biologically active fragment or derivative thereof or a small molecule that activates the signal pathways activated by RASSF8 can be administered to a subject in need thereof according to any formulations/regimens available to those of skill in the art including those discussed above for cells. For example, in certain embodiments medium or other suitable solution can comprise small molecules, RASSF8 protein or biologically active fragments or derivatives thereof.

Uses for the Cells

(1) Transplantation

Subjects in need for a stem cell (e.g. a HSC) transplantation could benefit from the cells produced by the methods of this invention. Subjects in need for such a transplantation include subjects suffering from non malignant blood disorders, particularly immunodeficiencies (e.g. SCID, fanconi's anemia, severe aplastic anemia, congenital hemoglobinopathies, and metabolic storage diseases such as for example, Hurler's disease, Hunter's disease, mannosidosis, among others) or cancer, particularly hematological malignancies such as acute leukemia, chronic leukemia (myeloid and lymphoid), lymphoma (Hodgkin's and non-Hodgkin's), multiple myeloma, myelodysplastic syndrome, or non-hematological cancers such as breast carcinoma, colon carcinoma, neuroblastoma, and renal cell carcinoma.

(2) Pharmaceutical Testing

Pharmaceutical testing is moving more and more from in vivo experimentation to in vitro studies. Since the availability of large numbers of stem cells (e.g. HSC) is insufficient for such testing, the methods of this invention can be used to produce large amounts of undifferentiated stem cells that could be used for screening for pharmaceutical compounds.

Accordingly, the cells of the invention can be used in such testing and screening methods.

(3) Development of Differentiation Methods

By screening for inhibitors of RASSF8 the cells produced by the methods of this invention can be used for developing new differentiation methods. Expression of RASSF8 can be downregulated or inhibited by methods well known in the art, e.g. by using small molecules or siRNA (small interfering RNA (siRNA; SiRNAs usually have a well defined structure: a short (about 21-nt) double-strand of RNA (dsRNA) with 2-nt 3′ overhangs on either end), sometimes known as short interfering RNA or silencing RNA, are a class of 20-25 nucleotide-long RNA molecules that play a variety of roles in biology. Most notably, this is the RNA interference pathway (RNAi) where the siRNA interferes with the expression of a specific gene, additionally, siRNAs play additional roles in RNAi-related pathways, e.g., as an antiviral mechanism or in shaping the chromatin structure of a genome).

Uses of the Protein/Nucleic Acid

One embodiment provides a method for maintaining/expanding stem cells in an undifferentiated state comprising culturing stem cells wherein RASSF8 is added as a protein or biologically active fragment or derivative thereof, or in the presence of a small molecule that activates the signal pathways activated by RASSF8. The protein and small molecules can further be use in the development of differentiation methods and pharmaceutical testing as described above.

Another embodiment provides a method of treatment comprising administering RASSF8 protein or DNA coding for RASSF8 protein, a biologically active fragment or derivative thereof or a small molecule that activates the signal pathways activated by RASSF8 to a subject in need thereof so as to increase stem cells in the subject, e.g., HSCs. Subjects in need of this type of treatment include subjects suffering from (e.g., afflicted with) non malignant blood disorders, particularly immunodeficiencies (e.g. SCID, fanconi's anemia, severe aplastic anemia, or congenital hemoglobinopathies, or metabolic storage diseases, such as Hurler's disease, Hunter's disease, mannosidosis, among others) or cancer, particularly hematological malignancies, such as acute leukemia, chronic leukemia (myeloid or lymphoid), lymphoma (Hodgkin's or non-Hodgkin's), multiple myeloma, myelodysplastic syndrome, or non-hematological cancers such as breast carcinoma, colon carcinoma, neuroblastoma, or renal cell carcinoma.

The present invention is additionally described by way of the following illustrative, non-limiting Example that provides a better understanding of the present invention and of its many advantages.

### Introduction

The transcriptome of two subpopulations of human CD34⁺ cells were compared (CD34⁺CD38⁻CD33⁻c-kit⁺Rho^{lo} (Rho^{lo}) cells enriched in SCID-repopulating cell (SRC) cells and CD34⁺CD38⁻CD33⁻Rho^{hi} (Rho^{hi}) cells, that do not contain SRC) from UCB and bone marrow (BM) to identify candidate genes that govern self-renewal of HSC. 277 genes were differentially expressed between Rho^{lo} and Rho^{hi} cells from both BM and UCB and performed a high through-put screen for the function of 80 of these genes by morpholino (MO) knockdown in Zebrafish. 16 genes were identified that caused a decreased blood phenotype: C12ORF2, CCR7, HSPC039, IRAK3, PRKCH, SPRY4, MGC15875, FOXM1, SUZ12, MAFB, FLJ14917, MRPS6, SLCO3A1, SNX5, CTBP2 and SPARC. Studies were initiated to confirm a role of these genes in mammalian hematopoiesis. Viral constructs were generated to force express and knockdown the target genes in murine (m)HSC, following which repopulation of HSC is evaluated in competitive repopulation studies in mice, and expansion of cKit⁺Lin⁻Sca1⁺ (KLS) cells (the phenotype of mHSC) in vitro.

C12ORF2/RASSF8 was identified from human cDNA libraries. C12ORF2 contains an RA domain, which lead to its renaming to RASSF8, even though it is not known that RASSF8 binds GTPases. There are 10 family members in RASSF protein family: RASSF1-RASSF6 contain, aside from the RA domain, an Sav/RASSF/Hpo (SARAH) domain mediating interactions with microtubules affecting cell cycle progression. RASSF1, most extensively studied, affects mitosis completion by interaction with microtubules, and is involved in Ras-mediated pro-apoptotic pathways. Although analysis of other RASSF family members has been more limited, many appear to also induce apoptosis in a number of cell lines or in developing Xenopus. Loss of expression of one or another RASSF family member, chiefly by promoter methylation, has been documented in a number of tumors. RASSF7-RASSF10, also termed NT-RASSF proteins, contain the RA domain but no SARAH domain. Little is known regarding RASSF8. RASSF8 is widely expressed as a 419 and 392 aa isoform (RASSF8a and RASSF8b). RASSF8 interacts with FRMD6, a cytoskeletal protein of unknown function, and PSMD4, a subunit of the 19S regulator of the 26S proteasome. Binding to known partners of other RASSF proteins has not yet been documented for RASSF8. Forced expression of RASSF8 inhibits anchorage-independent growth and reduces proliferation of lung carcinoma cells, suggesting a tumor-suppressor function for RASSF8. Finally, a reciprocal translocation between C12ORF2 and Fibulin-1 lays at the basis of a congenital syndrome of synpolydactyly.

The function of RASSF8 was evaluated in murine hematopoiesis. Using a retrovirus containing the RASSF8 cDNA (MSCV-RASSF8-IRES-GFP) RASSF8 was forced expressed in Lineage (Lin⁻) mBM cells, selected GFP/RASSF8⁺ cells by FACS and performed competitive repopulation studies in mice. Forced expression of RASSF8 significantly decreased hematopoietic reconstitution after 3 months compared with cells transduced with an empty virus. Moreover, fewer colony forming cells (CFC) grew from MSCV-RASSF8-IRES-GFP than MSCV-IRES-GFP transduced cells, even though colonies generated from MSCV-RASSF8-IRES-GFP transduced cells were immature blasts. Consistent with the notion that RASSF8 inhibits differentiation, significantly more KLS cells were present in cultures of MSCV-RASSF8-IRES-GFP than MSCV-IRES-GFP transduced cells.

The LMP retroviral vector wherein GFP expressed from an internal Pgk promoter was used to express an shRNA against RASSF8 from the LTR-promoter (LMP-shRNA-RASFF8). Lin⁻ mBM cells were transduced and GFP/shRASSF8⁺ cells selected by FACS. Results of competitive repopulation studies in mice are still pending, but significantly fewer CFC were present in methylcellulose assays of LMP-shRNA-RASSF8 than untransduced cells, and most Lin⁻ cells wherein RASSF8 was knocked down had acquired lineage markers by d3 after transduction.

These and studies wherein RASSF8 was force expressed in hematopoietic cell lines did not document apoptosis, which differs from what was documented for other RASSF family members.

Using a commercially available anti-RASSF8 Ab, it was shown that RASSF8 is localized chiefly in the cytoplasm of NIH-3T3 and WEHI cells, but can also be found in the cell nucleus. Immunoprecipitation with an anti-RASSF8 Ab is possible, and there is evidence that multiple proteins co-precipitate with RASSF8, visualized on a silverstained gel.

Thus, RASSF8 is one of the genes identified to be more highly expressed in Rho^{lo} than Rho^{hi} UCB and BM cells. MO-mediated knockdown in zebrafish inhibited hematopoiesis. Herein it is shown, by subsequent studies in mice, that forced expression of RASSF8 inhibits differentiation, whereas knockdown of RASSF8 using shRNAs induces accelerated differentiation.

### EXAMPLE I

### Material and Methods

Animals

8 to 10 week old C57BL/6J (CD45.2) female mice were used as recipient mice and B6.SJL-PTPRCA (CD45.1) male mice were used as donor mice. C57BL/6J mice were purchased from Janvier (Le Genest St Isle, France). B6.SJL-PTPRCA (CD45.1) mice were purchased from Jackson Laboratories (Bar Harbor, Me.). Mice were maintained at Research Animal at the Katholieke Universiteit Leuven. All experiments were approved by the Ethics Committee for Animal Research (KU Leuven, Belgium).

Isolation of Lin− Bone Marrow Cells

BM cells were isolated from femurs and tibia from CD45.1 mice. Lineage negative (Lin⁻) cells were obtained using the EasySep® Mouse Hematopoietic Progenitor Cell Enrichment Kit (Stem Cell Technologies) per manufacturer's protocol.

Construction of Retroviral Vectors

For the overexpression of genes in hematopoietic stem cells, the MIG (MSCV-IRES-GFP) overexpression retroviral plasmid was used, originally described by Dr. Keith Humphries (BCCRC, Vancouver, Canada). The coding region of Rassf8 gene was amplified by PCR using cDNA from Lin− cells. A reversed primer was used with an additional FLAG-tag for subsequent protein detection. Both primers contained restriction sites, to clone it into the MIG-plasmid. The forward primer contained an MfeI restriction site, whereas the reversed primer contained an XhoI site. The multiple cloning site of the MIG plasmid contained an EcoRI (same overhang than MfeI) and a XhoI site. Final plasmids were sent for sequencing to verify the sequence of the cloned Rassf8 gene (VIB genetic sequencing facility, University of Antwerp, Belgium).

Virus was made by triple transfection of the Rassf8-MIG transfer plasmid, together with the Gag-pol and VSVG plasmids. Cells were cultured in serum-free Optimem medium (Invitrogen). A first virus harvest was performed 48 h after transfection, a second harvest 72 h after transfection. Virus was concentrated using vivaspin 20 columns (MWCO=50000)(Sartorius). Thereafter, virus is kept at −80° C. Primers were ordered from Invitrogen.

| | |
|---|---|
| Primers: | |
| M-C12orf2F-total MfeI | |
| GATATACAATTGCACCATGGAACTTAAAGTGTGGGTG | (SEQ ID NO:1) |
| M-C12orf2R-total XhoI | |
| CTTAAACTCGAGCTACTTATCGTCGTCATCCTT | (SEQ ID NO:2) |
| GTAATCTAGATAGATGCCTTCAGGATT A | |

Transduction of Lin− Cells

Lin− cells were plated in low attachment plates (Corning) at a density of 50,000 cells/ml and were cultured for 24 hours in prestimulation medium consisting of serum-free expansion medium (Stem Cell Technologies) supplemented with 100 ng/ml mTpo, 100 ng/mL Flt3L 100 ng/ml mSCF, and 30 ng/ml IL3. After 24 hours cells were transduced with the MIG or Rassf8-MIG vector using an MOI of 5. Infection was done by spinfection at 800×g for 1 h. Afterwards, cells were transferred back to low attachment plates. Cells were transduced a second time using the identical method at 48 hours after plating.

72 h after initial plating, Lin− GFP+ cells were selected by FACS after they were labeled with APC-coupled antibodies against hematopoietic lineage markers (BD biosciences). For the non-transduced control, cells were selected based on Lin− characteristics alone.

Cell Culture

### Cell Expansion Culture

100000 Lin− cells were plated per well of a 6-well low attachment plate (Corning) in 2 ml of serum-free expansion medium (Stem Cell Technologies) supplemented with 100 ng/ml mTpo, 100 ng/mL Flt3L, 100 ng/ml mSCF, and 30 ng/ml IL3. Cells were cultured for 8 days at 37° C. with 5% CO₂.

### Colony-Forming Cell (CFC) Assay

Fresh or culture progeny were plated in methylcellulose medium supplemented with mSCF, mIL-3, mIL-6 and Epo (M3434, Stem Cell Technologies). All cultures were incubated at 37° C. and 5% CO₂. Colonies were counted between day 10 and 12.

### Transplantation Assays

For competitive repopulation studies, CD45.2 recipient mice were irradiated with 10 Gy 20 hours prior to transplantation. 10⁵ BMMN cells from CD45.2 mice were mixed with 25×10³ CD45.1 untransduced cultured Lin− cells, MIG transduced or Rassf8-MIG transduced Lin− GFP+ cells. A recipient mouse was considered multi-lineage repopulated if the percentage of donor cell-derived cells (CD45.1) was >1% and donor cells contributed to all three hematopoietic lineages (myeloid, T lymphoid and B lymphoid cells) in peripheral blood and/or bone marrow. Blood engraftment levels were evaluated at 4 weeks and 12 weeks.

### Antibodies:

| Company | Catalog nr | Product |
|---|---|---|
| BD | 558701 | APC CD 45.1 Mouse |
| BD | 550882 | APC Mouse IgG 2A |
| BD | 558074 | APC Mouse Lineage Cocktail |
| | | Antibody + Isotype |
| BD | 553989 | PE Rat IgG 2B |
| BD | 552950 | PercPCy5.5 mouse CD 45.2 |
| BD | 550927 | PercPCy5.5 mouse IgG 2A |
| Ebioscience | 12 1171 82 | PE ckit Mouse |
| Ebioscience | 12-4031-82 | PE Rat IgG 2B |
| Ebioscience | 45 4321 80 | PercP Rat IgG 2A |
| Ebioscience | 45 5981 82 | PercP Sca1 Mouse |
| Ebioscience | 00-6993 | 7-AAD viability staining solution |

Quantitative RT-PCR

Total RNA was harvested from Lin− cells using the Qiagen RNeasy Kit (Qiagen, Hilden, Germany). 2 μg of total RNA used for cDNA synthesis using the SuperScript™ Double-Stranded cDNA Synthesis Kit (Invitrogen). Q-RT-PCR on the cDNA was performed using the Platinum® SYBR® Green qPCR SuperMix-UDG kit (Invitrogen). Q-RT-PCR was carried out using a realplex mastercycler (Eppendorf, France). Primers were ordered from Invitrogen.

Primers:

(SEQ ID NO:3)

| | |
|---|---|
| Mm-GAPDH-RT-F | GAAACCTGCCAAGTATGATGAC |

(SEQ ID NO:4)

| | |
|---|---|
| Mm-GAPDH-RT-R | TTGTCATACCAAGGAAATGAGC |

(SEQ ID NO:5)

| | |
|---|---|
| Mm-RASSF8-endoRT-F | CCAAGCTATAGGTCGAACTG |

(SEQ ID NO:6)

| | |
|---|---|
| Mm-RASSF8-endoRT-R | GTCGATACAAAGTTCTTTCAGG |

(SEQ ID NO:7)

| | |
|---|---|
| Mm-RASSF8-exoRT-F | TAGAGCAGTTGACCAAAGAG |

(SEQ ID NO:8)

| | |
|---|---|
| Mm-RASSF8-exoRT-R | CTACTTATCGTCGTCATCCT |

### Statistical Analysis

Unequal Variance T-Test

Results

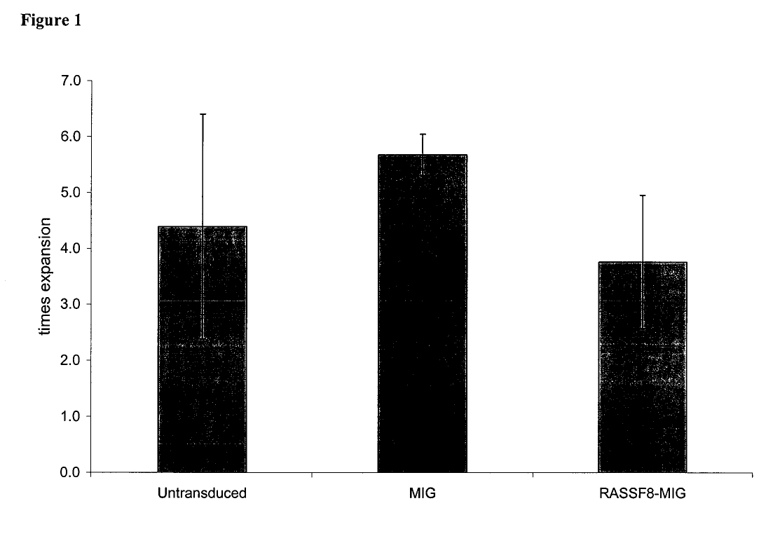

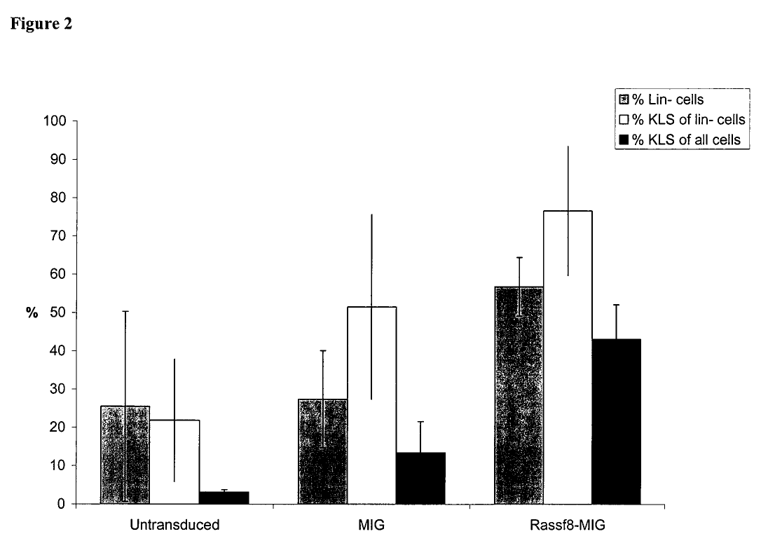

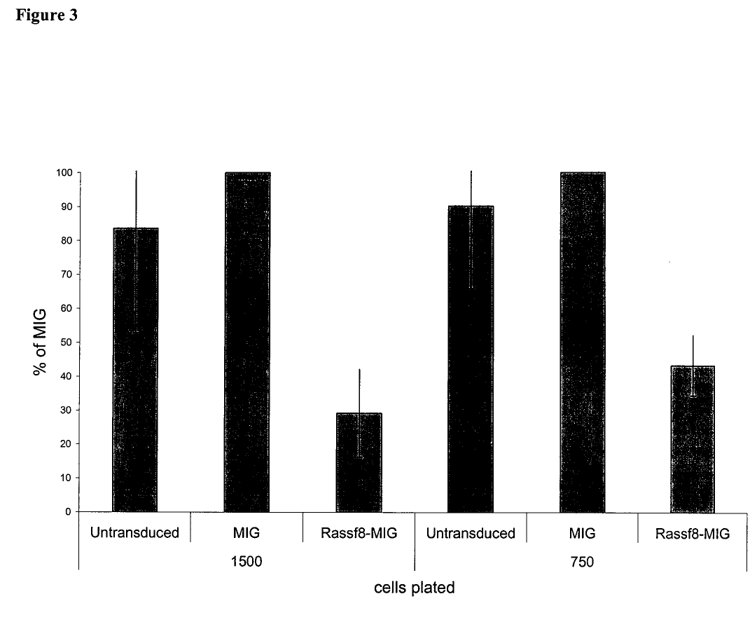

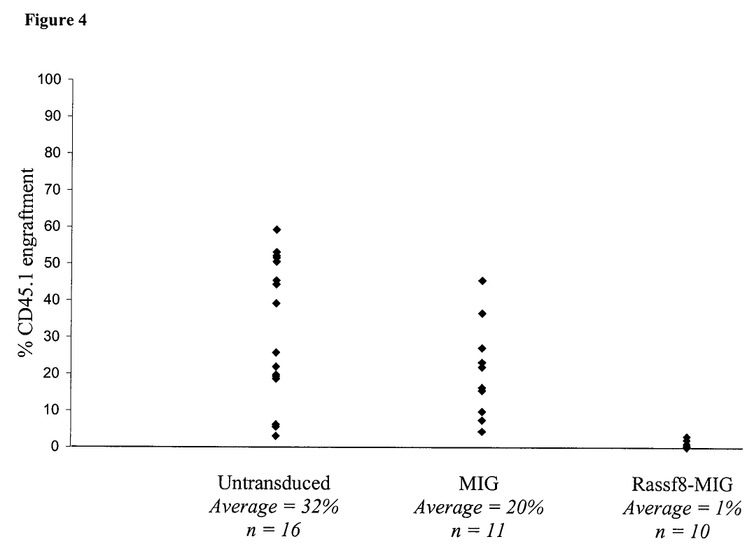

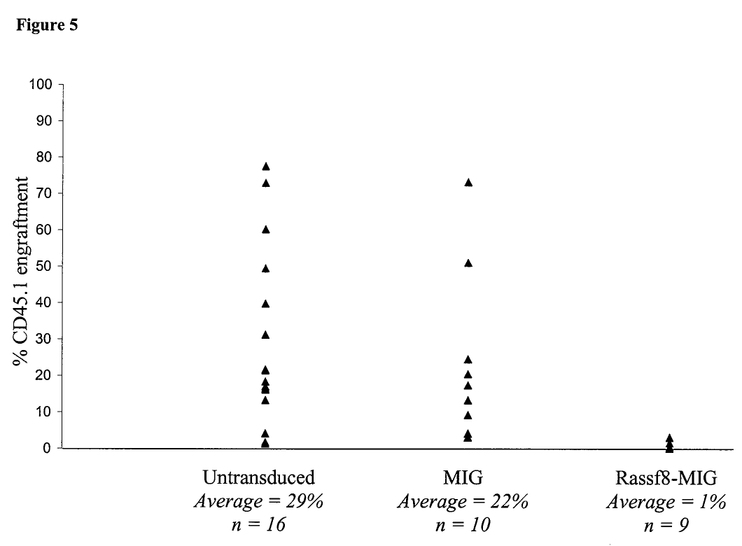

To test the function of RASSF8 in mammalian hematopoiesis, RASSF8 cDNA was overexpresed in lineage depleted murine bone marrow cells (Lin−) by retroviral transduction using the MSCV-RASSF8-IRES-GFP vector (rMIG-RASSF8). Sorted transduced GFP+ cells were cultured in serum-free medium (supplemented with SCF, TPO, Flt3L and Il-3) for three to five days. No significant differences were seen in overall cell expansion, cell death (7-AAD staining) or cell proliferation (thymidine incorporation assay and propidium iodide staining) between rMIG-RASSF8 transduced cells and cells transduced with the control vector (rMIG) (FIG. 1). However, it is noted that a significant accumulation of the stem cell enriched cKit+lin−Sca1+ (KLS) population in rMIG-RASSF8 transduced cells (43+/−9%) compared to control cells transduced with rMIG (13+/−8%, p=0.003; n=4) after five days (FIG. 2). The total number of colony forming cells (CFCs) produced by 750 rMIG-RASSF8 transduced Lin− cells (43+/−9) was significantly lower than in rMIG transduced cells (90+/−24, p=0.008; n=3)(FIG. 3). CFCs from rMIG-RASSF8 transduced cells were morphologically smaller and more compact, consistent with the morphology of primitive blast-forming colonies. Competitive repopulation assays using 25 000 rMIG-RASSF8 or rMIG transduced CD45.1 Lin− cells versus 100 000 CD45.2 mononuclear BM cells were performed to evaluate HSC engraftment potential. Four weeks after transplantation only 1% CD45.1 cells were found in the peripheral blood (PB) of the animals receiving rMIG-RASSF8 transduced cells vs. 20% CD45.1 cells in animals that had received rMIG transduced cells (p<0.001) (FIG. 4). Likewise, PB analysis at 3 months demonstrated nearly no reconstitution of rMIG-RASSF8 transduced CD45.1 cells (1%) vs 22% rMIG transduced CD45.1 cells (p<0.001) (FIG. 5).

Thus, RASSF8 blocks hematopoietic differentiation and short term overexpression of RASSF8 leads to HSC expansion. When RASSF8 is persistently overexpressed, the inability of HSC to differentiate prevents to the assessment of HSC frequency and engraftment, i.e. generation of mature blood cells. Permanent universal overexpression or loss of RASSF8 will lead to embryonic lethality, due to major impairment of the hematopoietic system.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method for maintaining/expanding stem cells in an undifferentiated state comprising culturing stem cells wherein RASSF8 is overexpressed or added as a protein or biologically active fragment or derivative thereof, or in the presence of a small molecule that activates the signal pathways activated by RASSF8.

2. The method of claim 1, wherein RASSF8 is overexpressed or added for a period of about 1 day to about 14 days.

3. The method of claim 1, wherein the stem cells are isolated from umbilical cord, bone marrow, placenta, muscle, endothelium, bone, central nervous system (CNS), liver, gastrointestinal track, lung, blood or skin.

4. The method of claim 1, wherein the stem cells are HSCs.

5. The method of claim 1, wherein the stem cells are of human origin.

6. Cells produced according to the method of claim 1.

7. A pharmaceutical composition comprising the cells of claim 6.

8. A method of treatment comprising administering a therapeutically effective amount of the cells of claim 6 to a subject in need thereof.

9. The method of treatment of claim 8, wherein the subject in need thereof is suffering from a non-malignant blood disorder.

10. The method of treatment of claim 9, wherein the non-malignant blood disorder is selected from the group of immunodeficiencies comprising SCID, fanconi's anemia, aplastic anemia, congenital hemoglobinopathy, or metabolic storage disease.

11. The method of treatment of claim 10, wherein the metabolic storage disease is Hurler's disease, Hunter's disease, or mannosidosis.

12. The method of treatment of claim 8, wherein the subject in need thereof is suffering from cancer.

13. The method of treatment of claim 12, wherein the cancer is selected from the group of hematological malignancies comprising acute leukemia, chronic leukemia, lymphoma, multiple myeloma, myelodysplastic syndrome, or non-hematological cancer.

14. The method of treatment of claim 13, wherein the chronic leukemia is myeloid or lymphoid.

15. The method of treatment of claim 13, wherein the lymphoma is Hodgkin's or non-Hodgkin's lymphoma.

16. The method of treatment of claim 13, wherein the non-hematological cancer is breast carcinoma, colon carcinoma, neuroblastoma, or renal cell carcinoma.

17. The method of treatment of claim 8, wherein the subject has been treated with chemotherapy or radiation.

18. The method of claim 17, wherein the subject has lost HSCs.

19. A method of treatment comprising administering RASSF8 protein, a biologically active fragment or derivative thereof or a small molecule that activates the signal pathways activated by RASSF8 to a subject in need thereof.

20. The method of claim 19, wherein the subject is afflicted with a non-malignant blood disorder or cancer.

21. The method of claim 19, wherein the RASSF8 protein, a biologically active fragment or derivative thereof or a small molecule that activates the signal pathways activated by RASSF8 increases HSCs in the subject.


SEQUENCE LISTING (10 sequences)

SEQ ID NO: 1 - DNA, 37 bp, Artificial Sequence
  a synthetic oligonucleotide
gatatacaattgcaccatggaacttaaagtgtgggtg

SEQ ID NO: 2 - DNA, 61 bp, Artificial Sequence
  a synthetic oligonucleotide
cttaaactcgagctacttatcgtcgtcatccttgtaatctacatagatgccttcaggatta

SEQ ID NO: 3 - DNA, 22 bp, Mus musculus
gaaacctgccaagtatgatgac

SEQ ID NO: 4 - DNA, 22 bp, Artificial Sequence
  a synthetic oligonucleotide
ttgtcataccaaggaaatgagc

SEQ ID NO: 5 - DNA, 20 bp, Mus musculus
ccaagctataggtcgaactg

SEQ ID NO: 6 - DNA, 22 bp, Mus musculus
gtcgatacaaagttctttcagg

SEQ ID NO: 7 - DNA, 20 bp, Mus musculus
tagagcagttgaccaaagag

SEQ ID NO: 8 - DNA, 20 bp, Artificial Sequence
  a synthetic oligonucleotide
ctacttatcgtcgtcatcct

SEQ ID NO: 9 - DNA, 2330 bp, Homo sapiens
agcggaggcggcgcggggacgggcgctgggcggccgcggagctccgggtgccgccgcgtccccagcgccccggccggcccctctgggcggcctgcgggggcggcgcagttgcgaaactgagtgcctgtgtagatcatcctagaatacctgtgtggtgctgtccttcctcaagactacctcattctcactggctcggagactcctgcctgctgagctgactacacagacttagtcttctccactccgtgttcctgcagctagagacatgacctaacaccctgatgaccactctcagggaccttgagtgactggccggtgcaccatggaacttaaagtatgggtggatggagttcagaggattgtttgtggagtcactgaagtcacaacttgccaggaggttgtcatagccttagctcaagcaataggtcgaactggaaggtacacccttatagagaaatggagagatactgaaagacacttagcacctcatgaaaatcctatcatatccttaaacaaatgggggcagtatgctagtgatgtgcagctcattctacgacgaactgggccgtctctcagtgagcgacccacttcagacagtgtggctcgaattcctgaaagaactttatacaggcagagtctgccccccttagctaaactgaggcctcagattgacaaatcaatcaaaaggagggaaccgaaaaggaaatcactgacatttacaggaggtgccaaaggattaatggacatttttggaaaaggtaaagaaactgagtttaagcaaaaggtgctgaataactgcaaaacaacagcagatgagttgaagaagctaatccgtctgcagacagagaagcttcaatccattgagaaacagctggaatctaatgaaatagaaataagattttgggagcaaaagtataattccaaccttgaagaggaaattgtccgtctagagcaaaagatcaaaagaaacgatgtagaaattgaggaggaagaattctgggaaaatgaattacagattgaacaggaaaatgaaaaacagctgaaggatcaacttcaagaaataagacagaaaataacagaatgtgaaaacaaattaaaggactatttggcacagatccggactatggaaagtggtcttgaagcagaaaaattgcaacgggaagttcaagaggcacaggtcaatgaggaagaggttaaaggaaagatcggtaaggtcaaaggggagattgacattcaaggccagcagagtctgaggttggaaaatggcatcaaagctgtggaaagatctcttggacaagccaccaaacgcttacaggacaaagaacaggaactggagcagttgactaaggagttgcggcaagtcaatctccagcagttcatccagcagacagggacaaaagttaccgttttgccagcggagcccattgaaatagaggcctcacatgcagacattgaaagggggatcatcattctttctgataagcaggagtgtaaagattagatatcacaccaaaagctcaggcaagaaaagcaaaaatgaacaagtgggactacattaaacccaaaactcctgcacaacaaaggaaacgatcaccaaaatgaaaaggtggactgtagattgggagaaaatatttgcaaaccgttatctggtaggggtttaatatccaaactatataaggaactcacagaacttaaagaaaacaaataatccaattaaaaatggtcaaaggggctgggcatggtggctcacgcctgtaatctcagcactttgggaggccgaggtgggagaatcacaaggtcaggagactgagaccatcctggctaacaccatgatgggctaacacccatctctactaaaaatacgaaacattagccaggtgtggtggcacgcgcctgtaatcccagctactcgggaggctgaggcagaaggattgcttgaacccgggaggtggaggttgcagtgagccgagatcgcattactgcactccagcctgggtaacagagtgagactccatctcaaaaaaaaaaaaaaaatagatatttttccaaagaagacataaaaacagccaacaggtatatgaaaaagtgctcaacatcactgatcagcagggaaatgcaaatcaaagccacaatgagataccacctcacacctgttaggttacatctgctattgtcaaaaagataagagatagcaagtgtcagcaagggtctggagaaaagggaatttttgtatactgttggtgggaatgtaaatcagtacggctattgtggaaaacagtgtagaggtttctcaaaaaattaaaactatcatacaacccagcaaaaaaaaa

SEQ ID NO: 10 - protein, 392 aa, Homo sapiens
MELKVWVDGVQRIVCGVTEVTTCQEVVIALAQAIGRTGRYTLIEKWRDTERHLAPHENPIISLNKWGQYASDVQLILRRTGPSLSERPTSDSVARIPERTLYRQSLPPLAKLRPQIDKSIKRREPKRKSLTFTGGAKGLMDIFGKGKETEFKQKVLNNCKTTADELKKLIRLQTEKLQSIEKQLESNEIEIRFWEQKYNSNLEEEIVRLEQKIKRNDVEIEEEEFWENELQIEQENEKQLKDQLQEIRQKITECENKLKDYLAQIRTMESGLEAEKLQREVQEAQVNEEEVKGKIGKVKGEIDIQGQQSLRLENGIKAVERSLGQATKRLQDKEQELEQLTKELRQVNLQQFIQQTGTKVTVLPAEPIEIEASHADIERGIIILSDKQECKD